(19) United States
(12) Patent Application Publication
(10) Pub. No.: US 2007/0154514 A1
(43) Pub. Date: Jul. 5, 2007

(54) Therapeutic Structures

(76) Inventors: John J. Demakas, Spokane, WA (US); Brent W. Johnston, Spokane, WA (US); Anderson Collins, Corinth, TX (US)

Correspondence Address: WELLS ST. JOHN P.S., 601 W. FIRST AVENUE, SUITE 1300, SPOKANE, WA, 99201, US

(21) Appl. No.: 11/562,728

(22) Filed: Nov. 22, 2006

Related U.S. Application Data
(63) Continuation-in-part of application No. 11/322,821, filed on Dec. 30, 2005.
(60) Provisional application No. 60/844,954, filed on Sep. 14, 2006.

(51) Int. Cl. A61F 2/28

(52) U.S. Cl. 424/423; 623/16.11

(57) ABSTRACT

The invention includes skeletal support structures. The structures can be spinal plates (such as cervical plates), rods, hooks, vertebral spacers, vertebral structural replacement, joint replacement prosthetics, or screws; and can be formed of a carbon/metal matrix encapsulated with pyrocarbon. The screws and/or hooks can contain pores configured to receive growing bone to enhance union of the screws and/or hooks with skeletal material. The invention also includes therapeutic constructions containing structures attached to vertebrae through fasteners.

### RELATED PATENT DATA

This patent resulted from a continuation-in-part application of U.S. patent application Ser. No. 11/322,821, which was filed Dec. 30, 2005; and is related to a U.S. Provisional Application entitled “Therapeutic Structures”, which was filed Sep. 14, 2006, and which is Ser. No. 60/844,954.

### TECHNICAL FIELD

The invention pertains to therapeutic structures.

### BACKGROUND OF THE INVENTION

Numerous structures have been developed for therapeutic attachment to skeletal regions. Such structures can include, for example, various screws, hooks, plates, pins, cages and rods. Therapeutic uses of such structures can include, for example, temporary support to mobilize a skeletal region during healing in response to injury (for instance, screws, hooks, rods and/or plates utilized to mobilize a fractured bone during healing of the fracture), permanent support to replace a skeletal segment (for example, a knee or hip replacement), or permanent support to provide additional support beyond that offered by a skeleton region compromised by injury, disease, aging or genetic defect (for example, spinal plates, cages, hooks and rods provided for additional support beyond that offered by a deteriorated spine). Therapeutic structures also include structures utilized to attach tendons and/or ligaments to skeletal regions, such as, for example, various screws and washers.

It can be desired for therapeutic structures to have high biocompatibility, high strength, low weight, and good durability. Further, each type of structure can have particular demands for shape and performance imparted by its intended application. For instance, cervical plates are frequently placed between the spine and the esophagus within the neck of a patient. It is common for a cervical plate to be thick enough that a patient is aware of the plate during swallowing due to some interference of the plate with the esophagus. It is desired to create medical devices which are small enough that patients are completely unaware of the devices after the devices are in place. Presently, cervical plates are typically at least 1.5 millimeters (mm) thick, and it is desired to develop cervical plates which can be thinner while still providing sufficient support.

It is also desired to develop other improved therapeutic structures (for instance, screws, hooks, plates, pins, cages, rods, etc.) having biocompatibility, durability and high strength-to-weight ratio.

### SUMMARY OF THE INVENTION

In one aspect, the invention includes a therapeutic structure comprising a pyrocarbon-coated material. The therapeutic structure can be, for example, a screw, hook, washer, plate, cage or prosthesis.

In one aspect, the invention includes a therapeutic structure comprising a carbon-metal matrix. The carbon/metal matrix can be, for example, a tungsten/graphite matrix. The carbon/metal matrix can be at least partially covered with pyrocarbon.

In one aspect, the invention includes a spinal plate comprising a carbon-metal matrix. The spinal plate can be a cervical plate in some aspects of the invention, and in particular aspects of the invention the carbon/metal matrix can comprise a tungsten/graphite matrix.

In one aspect, the invention includes a cervical plate that is less than or equal to about 1.5 mm thick. The plate can comprise a carbon/metal matrix. Alternatively, or additionally, the plate can be coated with pyrocarbon.

In one aspect, the invention includes a therapeutic construction. The construction comprises a segment of a spinal column containing a pair of vertebrae and a disk between the vertebrae. The construction also comprises a carbon/metal matrix structure attached to each vertebra of the pair of vertebrae with fasteners. In some aspects, the fasteners can be screws, and in particular aspects such screws can have pores (or slots) extending therein.

In one aspect, the invention includes a screw configured to directly engage a bone. The screw comprises a shaft that is at least partially threaded, and comprises at least one pore extending into the shaft and configured to receive growing bone structure to enhance union of the screw with bone. The screw can be of a composition comprising a carbon/metal matrix. Alternatively, or additionally, the screw can be coated with pyrocarbon.

In one aspect, the invention includes a hook configured to engage a bone. The hook can be of a composition comprising a carbon/metal matrix. Alternatively, or additionally, the hook can be coated with pyrocarbon.

### BRIEF DESCRIPTION OF THE DRAWINGS

Preferred embodiments of the invention are described below with reference to the following accompanying drawings.

FIG. 1 is a diagrammatic, top view of an exemplary spinal plate in accordance with an aspect of the present invention.

FIG. 2 is a diagrammatic, cross-sectional view along the line 2-2 of FIG. 1.

FIG. 3 is a diagrammatic, fragmentary view of an assembly comprising the plate of FIG. 1 attached to a pair of segments of a spinal column.

FIG. 4 is a diagrammatic view of an exemplary screw in accordance with an aspect of the present invention.

FIG. 5 is a diagrammatic view of another exemplary screw in accordance with an aspect of the present invention.

FIG. 6 is a diagrammatic view of an assembly comprising a spine and implant constructions attached to the spine, in accordance with an aspect of the present invention.

FIG. 7 is a cross-section along the line 7-7 of FIG. 6.

FIG. 8 is a diagrammatic side view of a disassembled pedicle screw assembly, in accordance with an aspect of the present invention.

FIG. 9 is a diagrammatic side view of a pedicle screw in accordance with another exemplary aspect of the present invention.

FIG. 10 is a cross-sectional side view of the pedicle screw of FIG. 9, and specifically is a view along the line 10-10 of FIG. 9.

FIG. 11 is a diagrammatic, cross-sectional side view of another embodiment of a screw formed in accordance with an aspect of the present invention.

FIG. 12 is a diagrammatic, cross-sectional side view of a skeletal region having an exemplary screw of the present invention retained therein.

FIGS. 13-16 show various exemplary hooks that can be formed in accordance with aspects of the present invention.

FIG. 17 is a diagrammatic view of an exemplary spine cage embodiment.

FIG. 18 is a diagrammatic, cross-sectional side view of a skeletal region having an exemplary spine cage embodiment retained therein.

FIG. 19 is a diagrammatic view of an exemplary spinal spacer embodiment.

FIG. 20 is a diagrammatic view of a skeletal region having an exemplary spinal spacer embodiment associated therewith.

FIG. 21 is a diagrammatic view of a hip region having an exemplary hip treatment embodiment associated therewith.

FIG. 22 is a diagrammatic view of a shoulder region having an exemplary shoulder treatment embodiment associated therewith.

FIG. 23 is a diagrammatic view of an elbow region having an exemplary elbow treatment embodiment associated therewith.

FIG. 24 is a diagrammatic view of a knee region having an exemplary knee treatment embodiment associated therewith.

FIG. 25 shows a diagrammatic view of a screw and a washer, in accordance with an aspect of the present invention.

FIG. 26 shows a tendon attached to a bone in accordance with an aspect of the present invention.

FIG. 27 shows a replacement vertebral body embodiment.

### DETAILED DESCRIPTION OF THE PREFERRED EMBODIMENTS

This disclosure of the invention is submitted in furtherance of the constitutional purposes of the U.S. Patent Laws “to promote the progress of science and useful arts” (Article 1, Section 8).

The invention includes structures that can be utilized to provide support to skeletal regions. The structures can be utilized in veterinary applications for treating animals, or can be utilized for treating humans. In particular aspects, the invention includes spinal plates, such as, for example, cervical plates. In other particular aspects, the invention includes screws that can be inserted into bone. The screws can contain pores therein, with the pores being configured so that bone structure grows into the pores to improve union of the screws with bone. The bone structure growth into the pores can be enhanced by providing one or more bone-growth-stimulating compositions within the pores. Additionally, or alternatively, bone cement can be provided within the pores. In yet other aspects, the invention includes hooks that can attach to skeletal structures.

The various structures of the present invention can comprise carbon/metal matrices (in other words, can comprise matrices which include both carbon and metal). The carbon/metal matrices can comprise any suitable composition or combination of compositions. In some aspects, the carbon of the carbon/metal matrices can be in the form of graphite, and the metal can comprise a transition metal, such as, for example, a group 6 (new IUPAC notation) metal, such as tungsten. In exemplary aspects, the metal of a carbon/metal matrix of the invention can comprise, consist essentially of, or consist of tungsten. For instance, the carbon/metal matrices can consist essentially of, or consist of graphite/tungsten (with “graphite/tungsten” being understood to mean graphite and tungsten); with the tungsten being present to from about 1 weight % to about 30 weight %; typically from about 5 weight % to about 20 weight %; and most typically to about 10 weight %.

The carbon/metal matrices can be at least partially encapsulated with pyrocarbon (in other words, can be at least partially coated with a pyrolytic coating), or can be otherwise treated to form pyrocarbon that extends within the matrices and/or across surfaces of the matrices. Typically the carbon/metal matrices will be substantially entirely encapsulated, or even entirely encapsulated with pyrocarbon to enhance biocompatibility of the structures. The pyrocarbon can be provided to a thickness of at least about 0.01 inch; and can be formed as described in U.S. Pat. Nos. 5,514,410; 5,641,324; 6,217,616 and 5,843,183; and/or as available as On-X™ carbon from Medical Research Carbon Institute (MCRI™) of Austin, Tex. U.S.A.

In some aspects, structures described herein can be formed of any suitable pyrocarbon-coated material. The material can be, for example, a metal-containing material, such as a carbon/metal matrix of the type described above.

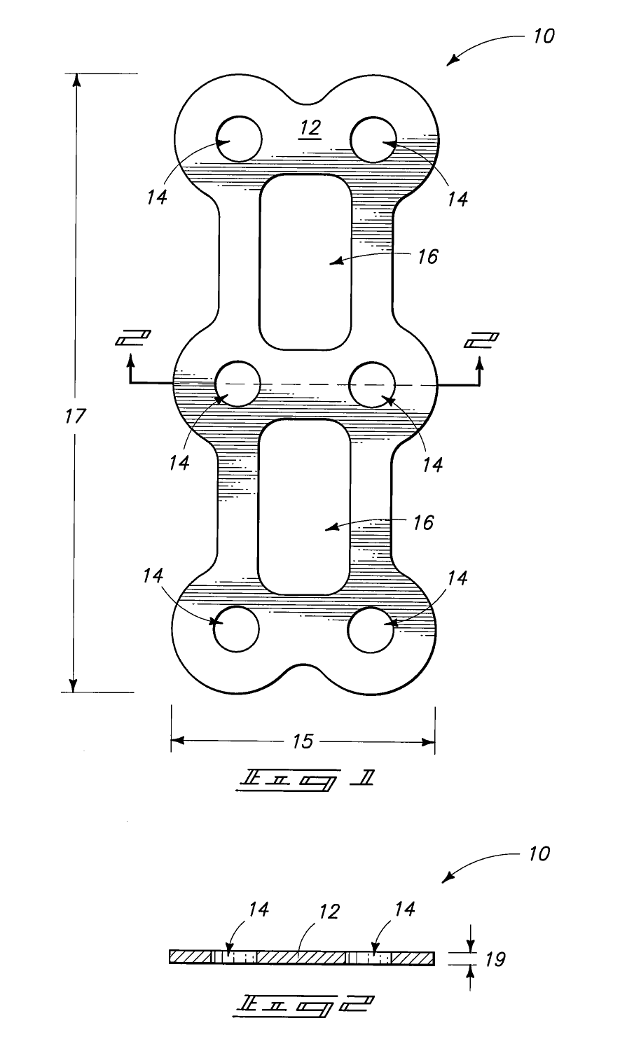

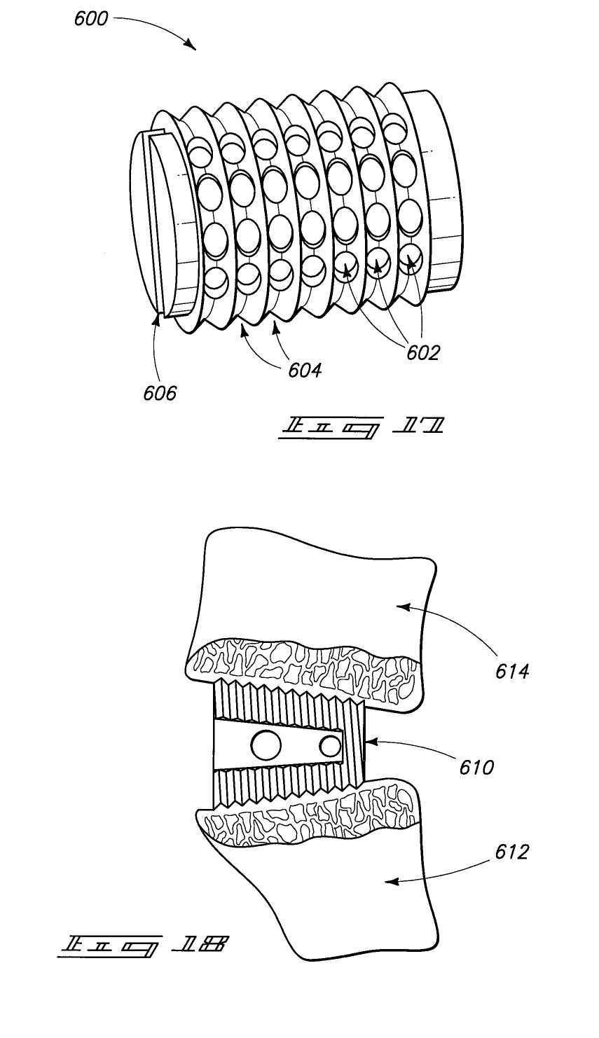

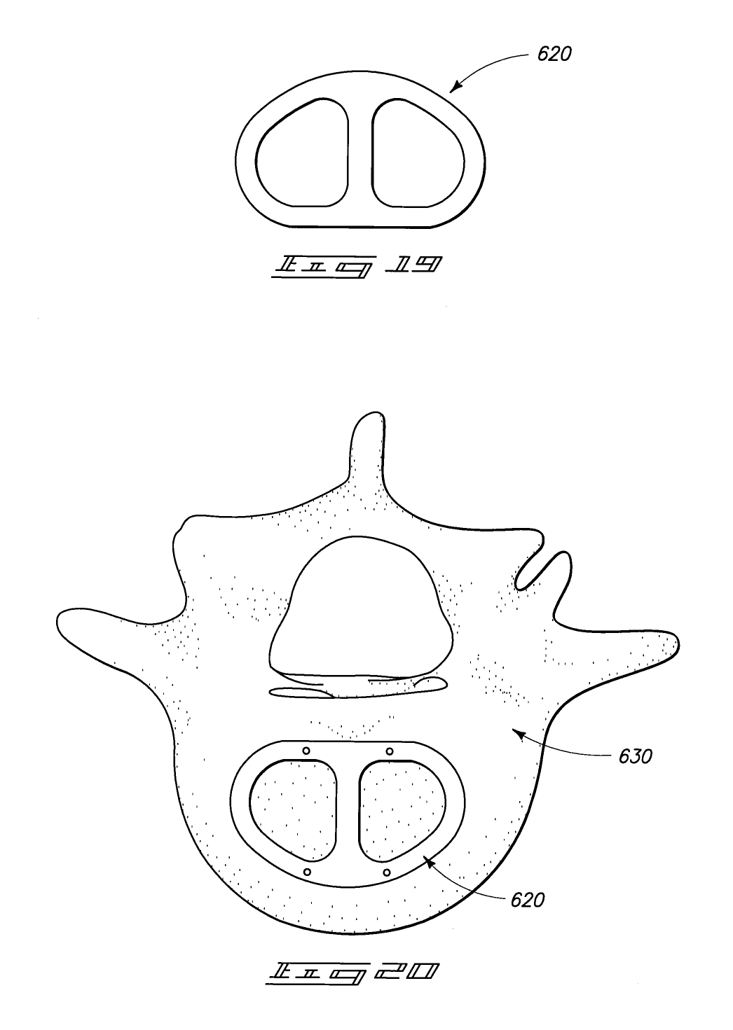

In some aspects, the present invention includes a recognition that the biocompatibility and strength-to-weight properties of pyrocarbon-coated metal/carbon matrices (for instance, the carbon/tungsten matrices discussed above) can be of particular advantage for utilization in screws, plates, hooks and other structures utilized for supporting, or replacing, skeletal regions; and/or for joining tissue (such as ligaments or tendons) to skeletal regions. Exemplary aspects of the invention are described below with reference to FIGS. 1-27.

Referring initially to FIGS. 1 and 2, such show an exemplary spinal plate 10 in accordance with an aspect of the present invention. Plate 10 comprises a structural material 12 having a plurality of holes 14 extending therethrough. The holes are configured for receiving screws, or other fasteners, ultimately utilized for retaining the plate to a spinal region. Additionally, a couple of windows 16 extend through the material 12, with such windows being configured to enable viewing of a bone graft provided within a spinal region behind plate 10. Persons of ordinary skill in the art will recognize that such windows are optional.

The shown plate is but one example of the numerous plates that can be utilized for attachment to spinal regions. It is to be understood that aspects of the present invention can be used for any spinal plate currently available, or which becomes available in the future. The spinal plate can be a cervical plate (in other words, can be configured for attachment to a cervical region of a spine); or can be configured for attachment to other regions of the spine (in other words, the thoracic region or lumbar region).

The material 12 of the spinal plate can comprise a pyrocarbon-coated material; and/or can comprise a carbon/metal matrix. In some aspects, material 12 can comprise, consist essentially of, or consist of a carbon/metal matrix; either alone, or coated with pyrocarbon. In some aspects, such carbon/metal matrix can comprise, consist essentially of, or consist of carbon and tungsten.

It can be advantageous for material 12 to comprise a carbon/metal matrix coated with pyrocarbon. The pyrocarbon can form a biocompatible coating. Additionally, the processing to form the pyrocarbon can significantly alter characteristics of the carbon/metal matrix to create much more strength within the carbon/metal matrix than would be present without such processing. Although the pyrocarbon is referred to as a coating, it is to be understood that the processing utilized to form the pyrocarbon can create changes throughout the carbon/metal matrix, as well as at the surface.

FIG. 1 shows plate 10 having a maximum width 15, and a maximum length 17; and FIG. 2 shows plate 10 having a maximum thickness 19. The maximum width and maximum length can be conventional. A typical conventional length will be from about 14 mm to about 90 mm, and a typical conventional width will be from about 10 mm to about 20 mm.

The maximum thickness 19 of plate 10 can be a conventional thickness, or in some aspects the plate 10 of the present invention can be significantly thinner than conventional devices due to the strength of the material utilized in the plate, and can, for example, be less than 1.5 mm, less than 1.2 mm, or even less than 1 mm.

The reduced thickness of plate 10 relative to conventional plates can eliminate prior art problems, such as, for example, the problem of patients feeling a cervical plate when they swallow.

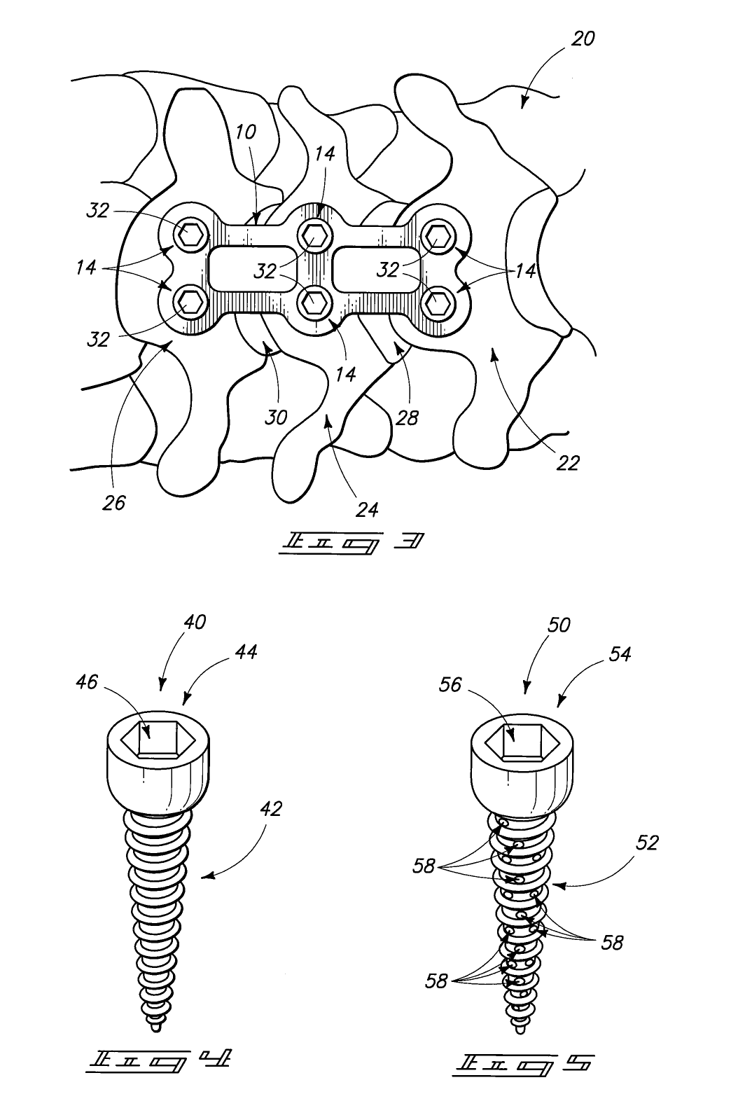

FIG. 3 shows a therapeutic construction comprising plate 10 joined to a region of a spinal column 20.

The spinal column includes a plurality of vertebra 22, 24 and 26, with intervening discs 28 and 30. Typically, a segment of the spinal column is understood to comprise a pair of vertebra and the disc between them; and accordingly the shown portion of the spinal column comprises two segments, with the vertebra 24 shared between the segments. The shown portion of the spinal column can correspond to any region of the spinal column, or in other words can comprise the cervical region, thoracic region or lumbar region of the spinal column.

Plate 10 is shown to extend across the two segments of the spinal column. It is to be understood that the invention also includes spinal plates which extend across only one segment of a spinal column, as well as including plates which extend across more than two segments of a spinal column.

Fasteners 32 are provided within the holes 14 of the spinal plate 10. The fasteners can be any suitable fasteners, including, for example, pins and screws. The fasteners can be conventional fasteners. However, in some aspects of the invention it can be preferred that the fasteners comprise pyrocarbon-coated material; and/or comprise a carbon/metal matrix. The fasteners can, for example, comprise, consist essentially of, or consist of a carbon/metal matrix, either alone, or coated with pyrocarbon. It can be advantageous to utilize a carbon/metal matrix coated with pyrocarbon for the reasons discussed above. In exemplary aspects, the fasteners comprise a pyrocarbon-coated matrix, with the matrix comprising, consisting essentially of, or consisting of carbon and tungsten.

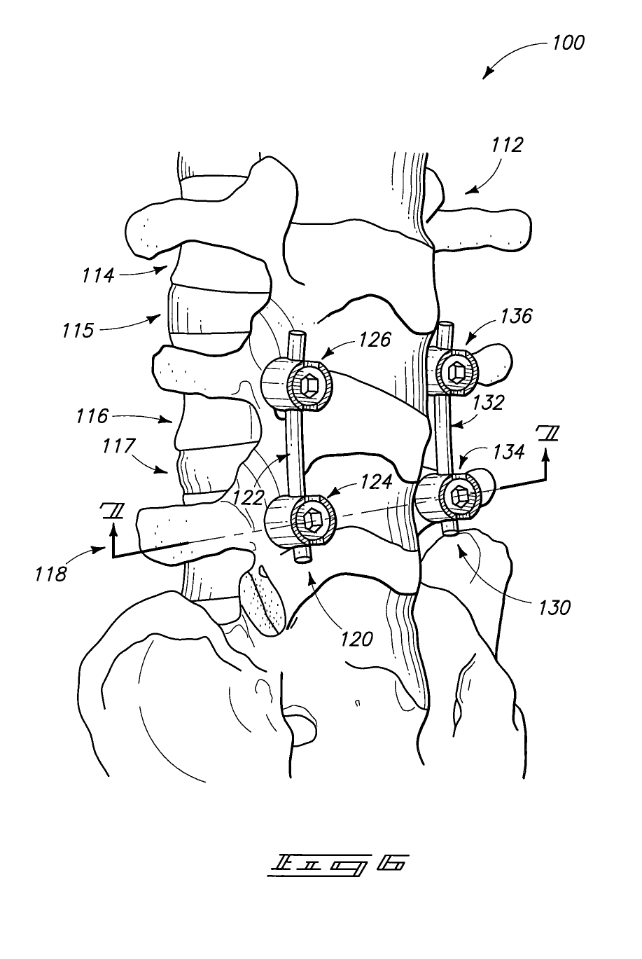

In some embodiments, the fasteners 32 will be screws. Exemplary screws are shown in FIGS. 4 and 5 as screws 40 and 50, respectively. Such screws can be formed of carbon/metal matrix coated with pyrocarbon. The screw 40 of FIG. 4 comprises a threaded shaft 42 joining to a head 44. The head has a tool-engagement slot 46 extending therein. The tool-engagement slot is configured to receive a tool utilized for screwing the screw 40 into a vertebra, and can correspond to a slot configured to receive any appropriate tool for screwing the screw into bone. The slot can be configured to receive, for example, a Phillips screwdriver or other cross-slotted screwdriver, a straight-slotted screwdriver, an Allen wrench, a Torx wrench, etc. The shown tool-engagement slot is configured for receiving a hexagonal-headed wrench.

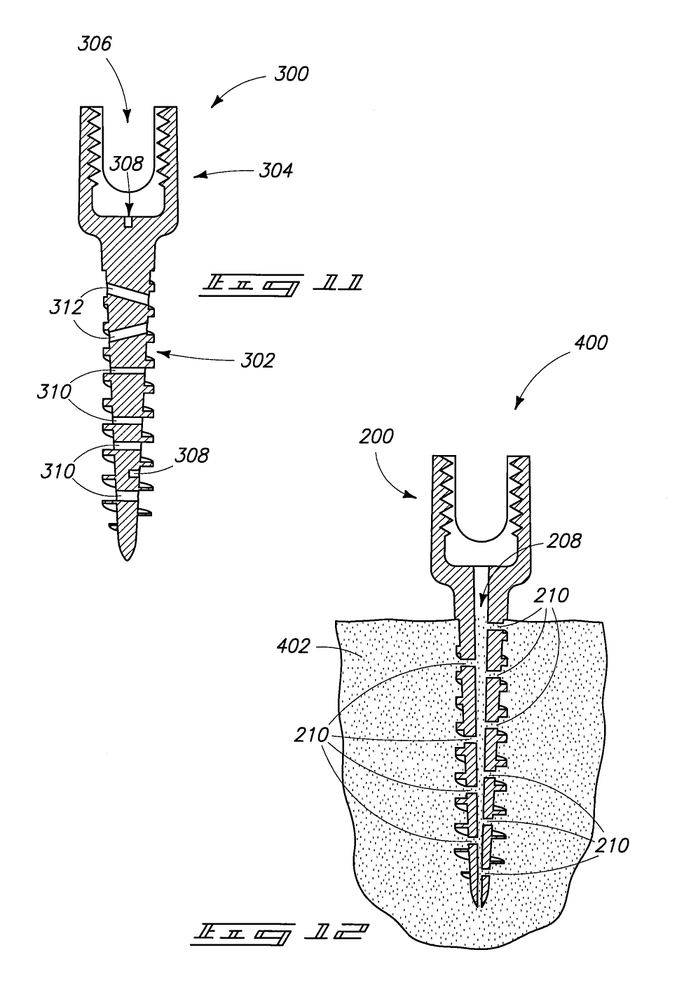

The screw 50 is similar to the screw 40, in that it comprises a threaded shaft 52, a head 50 for joining to the shaft, and a tool-engagement slot 56 extending within the head. However, screw 50 differs from screw 40 in that screw 50 comprises pores (or slots) 58 extending therein. Such pores can be similar to pores discussed below with reference to FIGS. 9-12, and accordingly can be configured for retaining bone cement and/or bone-growth stimulating material. Additionally, or alternatively, the pores can be configured so that bone can grow into the screw to enhance union of the screw with adjacent skeletal structure. Further, a cannula (not shown in FIG. 5, but discussed below with reference to FIG. 10) can extend longitudinally through screw 50.

A difficulty in attaching implant constructions to skeletal regions is that numerous conditions and diseases can lead to softened or weakened bone structures to which it is difficult to achieve robust union. For instance, osteoporosis increases bone porosity, which leads to softened bone structures. Implant constructions can frequently be screwed to osteoporotic bones in a problem-free manner. However, the screws holding the implant constructions to the bones can subsequently loosen from the bones through the normal forces exerted on the screws and implant constructions during ordinary day-to-day activities, or even can be pulled out of the bones if large forces occur.

Similar difficulties to those confronted with softened or weakened bone structures can also occur with normal, healthy bone structures.

In light of the problems confronted in obtaining and maintaining robust union of screws with bones, it can be preferred to utilize porous screws of the type shown in FIG. 5, instead of non-porous screws of the type shown in FIG. 4.

The plates discussed above are but one type of implant construction that can be attached to a skeletal region with screws. An exemplary procedure of utilizing screws to attach another type implant construction to a skeletal region is described with reference to FIGS. 6-8.

Referring to FIG. 6, such shows an assembly 100 comprising a spine 112 and a pair of implant constructions 120 and 130.

The spine comprises a series of vertebrae 114, 116 and 118 separated by disks 115 and 117.

The implant construction 120 comprises a rod 122 held between a pair of support structures 124 and 126; and the implant construction 130 comprises a rod 132 held between a pair of support structures 134 and 136. The rods 122 and 132 can be of any suitable composition or combination of compositions. In some aspects, the rods can comprise, consist essentially of, or consist of a carbon/metal matrix, either alone, or coated with pyrocarbon. In exemplary aspects, such carbon/metal matrix can comprise, consist essentially of, or consist of carbon and tungsten. In some aspects, the rods can comprise any suitable pyrocarbon-coated material.

The support structures 124, 126, 134 and 136 contain screws inserted into the pedicles of the vertebrae. In some aspects of the present invention, such screws can comprise, consist essentially of, or consist of a carbon/metal matrix, either alone, or coated with pyrocarbon. In exemplary aspects, such carbon/metal matrix can comprise, consist essentially of, or consist of carbon and tungsten. In some aspects, the screws can comprise any suitable pyrocarbon-coated material.

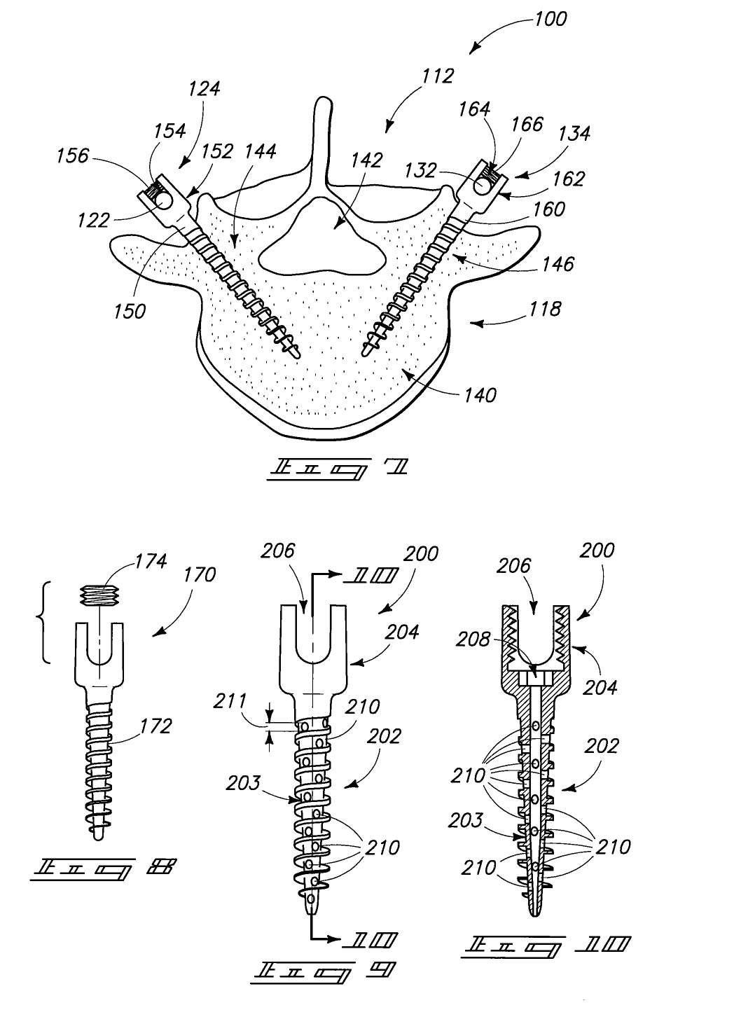

The screws have heads configured to enable retention of the rods. The support structures also comprise plugs inserted into the heads of the screws to lock the rods into the screws, as described in more detail below with reference to FIGS. 7 and 8. In some aspects, the plugs can comprise, consist essentially of, or consist of a carbon/metal matrix, either alone, or coated with pyrocarbon. In some aspects, the plugs can comprise any suitable pyrocarbon-coated material.

As mentioned above, a spinal segment is typically defined as a disc and the pair of vertebrae on opposing sides of the disc. Thus, the implant constructions 120 and 130 can each be considered to comprise a pair of pedicle screws on opposing sides of a spinal segment, and a rod joining the pedicle screws to one another.

FIG. 7 shows a cross-section through vertebra 118, and through support structures 124 and 134 of the constructions 120 and 130. The cross-section of FIG. 7 shows various anatomical features of vertebra 118, including the vertebral body 140, spinal canal 142 (through which the spinal nerve (not shown) passes), and pedicles 144 and 146. The cross-section of FIG. 7 also shows that support structures 124 and 134 comprise pedicle screws 150 and 160, respectively, which extend through pedicles 144 and 146, and into the vertebral body 140.

The pedicle screws 150 and 160 have heads 152 and 162, respectively. Such heads have channels 154 and 164 extending therein. The channels are configured to receive rods 122 and 132, and are further configured to receive plugs (or caps) 156 and 166 which retain the rods within the channels. The particular shown screws have threads within the channels. The threads within the channels receive threads of the plugs so that the plugs can be threadedly engaged within the channels to retain the rods. However, as will be recognized by persons of ordinary skill in the art, there are numerous other structural designs for pedicle screw heads which can be utilized for retaining rods to the pedicle screws. Also, persons of ordinary skill in the art will recognize that pedicle screws can be utilized for retaining other implant structures besides rods.

FIG. 8 shows a disassembled structure 170 comprising a pedicle screw 172 and a cap (or plug) 174. The screw 172 is identical to the screws 150 and 160 discussed above the reference to FIG. 7, and the cap 174 is identical to the caps 156 and 166. The disassembled structure of FIG. 8 shows that the cap is configured to threadedly engage within the channel in the head of screw 172.

As mentioned above, the invention includes aspects in which one or more pores are incorporated within screws. Such pores can be configured so that bone structure grows into the pores to improve the union of the screws with bone. The bone structure growth into the pores can be enhanced by providing one or more bone-growth-stimulating compositions within the pores.

FIGS. 9 and 10 show an exemplary screw 200 illustrating an aspect in which pores (or slots) are provided within the screw. The screw 200 is similar to the screws 150, 160 and 170 discussed above with reference to FIGS. 6-8. Accordingly, screw 200 comprises a threaded shaft 202 and a head 204 joined to the shaft. The shaft 202 is shown to be fully threaded, but it is to be understood that the shaft could also be only partially threaded in some applications. The screw 200 can comprise, consist essentially of, or consist of a carbon/metal matrix, either alone, or coated with pyrocarbon. In exemplary aspects, such carbon/metal matrix can comprise, consist essentially of, or consist of carbon and tungsten. In some aspects, the screw can comprise any suitable pyrocarbon-coated material.

The head 204 has a channel 206 extending therein. Such channel is threaded, as is apparent from the cross-sectional view of FIG. 10. The channel is configured so that a cap (or plug) can be utilized for retaining a rod within the channel. The screw 200 of FIGS. 9 and 10 can have any suitable dimensions of length and diameter.

The screw 200 of FIGS. 9 and 10 comprises a longitudinally-extending opening (also referred to herein as a cannula) 208 within the shaft, and a plurality of pores 210 (only some of which are labeled) extending through the shaft and to the opening 208. In some aspects, the shaft 202 can be considered to comprise a lateral sidewall 203, and the pores can be considered to extend through such lateral sidewall to the longitudinally-elongated opening 208. The pores and opening are configured to enable bone growth to extend into the screw 200.

Persons of ordinary skill in the art will recognize that a tool can be readily configured for inserting screw 200 into a bone.

The size of the longitudinally-elongated opening, size of the pores, and number of pores can vary depending on the intended application of screw 200. In some applications (discussed below with reference to FIG. 11), the longitudinally-elongated opening can be omitted. In some aspects of such applications, at least some of the pores can extend entirely through the screw (i.e., entirely from one lateral side of the screw to the opposing lateral side of the screw).

In applications in which the longitudinally-elongated opening is provided, the longitudinally-elongated opening can have any suitable length relative to the length of the shaft. In the shown application, the longitudinally-elongated opening is about the same length as the length of the shaft, but in other applications the longitudinally-elongated opening can be substantially shorter than the overall length of the shaft. Typically, however, if the longitudinally-elongated opening is provided within the shaft, the longitudinally-elongated opening will be at least about one third of the length of the shaft. The longitudinally-elongated opening can function to enable bone growth to extend within the screw, and in some applications (discussed below) the longitudinally-elongated opening can also be utilized for provision of bone-growth-stimulating compositions and/or bone cement. Alternatively, or additionally, the longitudinally-elongated opening can be utilized as a reservoir for retaining bone-growth-stimulating compositions and/or bone cement. In some aspects of the invention, it can be preferred that the longitudinally-elongated opening extend to the channel in the head, as shown, to enable bone-growth-stimulating compositions and/or bone cement to be injected into the longitudinally-elongated opening after the screw is at least partially inserted into a bone.

Regardless of whether or not a longitudinally-elongated opening is provided within the screw 200, there will be at least one pore (or cavity) extending into or through the wall of the shaft, and specifically through the bottom (i.e., tip) of the shaft and/or through a sidewall of the shaft. In the shown aspect of the invention, a pore extends through the bottom of the shaft, and several pores extend through the sidewall of the shaft. If the shaft is only partially threaded, one or more pores can extend into non-threaded portions of the shaft in addition to, or alternatively to, having one or more pores extending into threaded portions of the shaft.

Pores 210 can have any suitable size for enabling sufficient bone growth to occur within the pores to assist in retaining the screw to a bone. The shown pores are approximately circular along a lateral cross-section, with an exemplary pore having a cross-sectional diameter 211 of, for example, from about 0.1 mm to about 3 mm. The pores can extend through the sidewall 203 at any suitable angle. In some aspects, the pores will extend substantially orthogonally to a normal (i.e., longitudinal) axis of the screw, and in other applications at least some of the pores will extend at an angle which is not substantially orthogonal to the normal axis of the screw.

Although the screw of FIG. 10 is shown as a pedicle screw, it is to be understood that the screw can alternatively be another type of screw suitable for engaging bone. For instance, the screw can be a cervical screw, or a screw suitable for engaging regions other than the spine, including, for example, screws suitable for retaining hip implants, knee implants or shoulder implants; screws suitable for being utilized alone to retain bone fragments; screws suitable for attaching tendons or ligaments to skeletal regions; screws suitable for retaining various plates, cages and rods; or any other screws utilized for reconstruction, repair and/or support of skeletal regions. Any of such screws can comprise, consist essentially of, or consist of a carbon/metal matrix, either alone, or coated with pyrocarbon. Alternatively, any of such screws can comprise any suitable pyrocarbon-coated material.

FIG. 11 shows a screw 300 similar to the screw 200 discussed above with reference to FIG. 10, but lacking a longitudinally-extending cannula. Screw 300 comprises a threaded shaft 302 and a head 304 joined to the shaft. Screw 300 further comprises a threaded channel 306 extending into the head. Channel 306 has a slot 308 therein for receiving a tool which can be utilized for screwing the screw 300 into a bone. Such slot can be part of receptacle suitable for receiving, for example, a Phillips screwdriver or other cross-slotted screwdriver, a straight-slotted screwdriver, an Allen wrench, a Torx wrench, etc.

Screw 300 comprises pores 308, 310 and 312 analogous to the pores 210 associated with the screw 200 of FIGS. 9 and 10, with such pores being laterally-elongated openings in the embodiment of FIG. 11. The pores 308, 310 and 312 are configured for receiving bone structure grown into the pores. In the shown aspect, some of the pores extend entirely through the screw (specifically, pores 310 and 312) while one of the pores only extends partially into the screw (specifically, pore 308). Also, some of the pores are shown extending along approximately horizontal axes relative to a vertical axis defined by a normal axis of the screw (specifically, pores 310) while some of the pores are shown extending along axes tipped relative to such horizontal axes (specifically, pores 312).

Regardless of whether a porous screw is configured with a longitudinally-extending opening of the type shown in FIGS. 9 and 10, or without such longitudinally-elongated opening as shown in FIG. 11, bone-growth-stimulating material and/or various cements and bone adhering materials can be provided in one or more of the pores. For instance bone-growth-stimulating material can be provided to enhance growth of bone into the pores and/or polymethyl-methacrylate (PMMA) (a form of bone cement) can be provided within the pores to enhance adhesion to bone. If the longitudinally-elongated opening is present, the bone-growth-stimulating material and/or PMMA can be provided by injection of the bone-growth-stimulating material and/or PMMA through the longitudinally-elongated opening and into the pores joined to the opening before, after, and/or during screwing of the screw into bone. If the longitudinally-elongated opening is not present, the bone-growth-stimulating material and/or PMMA will typically be provided in the pores prior to screwing of the screw into the bone. Also, even if the longitudinally-elongated opening is present, the bone-growth-stimulating material and/or PMMA can be provided within the pores but not within the longitudinally-elongated opening, or vice versa. Further, if the longitudinally-elongated opening is present but some of the pores do not join with the opening, bone-growth-stimulating material and/or PMMA can be provided within the pores that do not join with the opening prior to screwing of the screw into the bone.

The bone-growth-stimulating material can comprise any composition or combination of compositions which stimulate bone growth. For instance, the bone-growth-stimulating material can comprise one or both of fibronectin and hydroxyapatite. Additionally, or alternatively, the bone-growth-stimulating material can comprise one or more bone morphogenetic proteins (bmp's) such as, for example, bmp2 and/or bmp7; and/or other osteo-inductive conductors. In some aspects, at least portions of the outer sidewall surfaces of the screw shafts (and particularly at least portions of the threaded surfaces of the shafts) are coated with one or both of fibronectin and hydroxyapatite to enhance union of the screws to bone. Such coating can be utilized in addition to the provision of bone-growth-stimulating material and/or bone cement in the pores and/or cannula of porous screws.

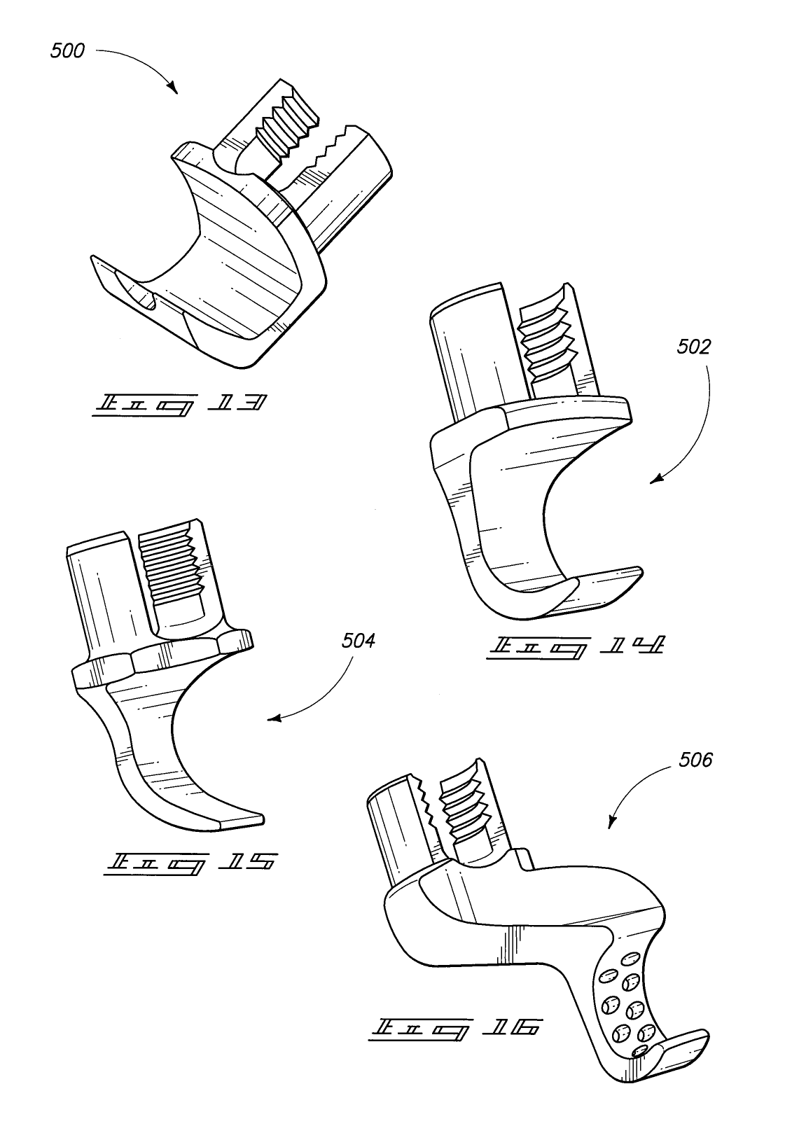

FIG. 12 shows an assembly 400 comprising the screw 200 (described above with reference to FIGS. 9 and 10) embedded in a bone 402. Structure, or matrix, of the bone is shown extending into the pores 210 of the screw, and also into the longitudinally-elongated opening 208. The bone structure within the pores and longitudinally-elongated opening enhances union of the screw with the bone. Such can alleviate prior art problems of screw loosening and screw pullout that could otherwise occur. Advantages of having bone growing into pores associated with a screw can occur in numerous applications, but can be particularly significant for patients suffering from bone-weakening ailments such as, for example, osteopenia or osteoporosis.

The shown screw 200 is a pedicle screw, and in the diagram of FIG. 12 bone has grown into the pores of the screw prior to assembly of the spine-stabilizing implant that is ultimately to be retained by the screw (specifically, prior to provision of rods and plugs of the type described with reference to FIG. 6). This can be a preferred aspect of the invention. Specifically, a porous screw can be fastened to a bone, and then left attached to the bone for a period of time sufficient to have bone growth extend into pores of the screw prior to attachment of an implant construction to the screw. This can enable the screw to become tightly joined with the bone through the growth of bone structure into the pores associated with the screw prior to providing stresses on the screw associated with an attached implant construction.

In the case of pedicle screws, for example, significant stresses can be applied to the screws once that rods are tightly joined to the screws. Such stresses can cause the screws to pull out of the pedicles if the stresses occur before a strong union of the screws with the pedicles has been achieved. Accordingly, it can be advantageous to wait until bone matrix material has grown into the pores of the pedicle screws (and in some aspects adhered to a surface of the screw) before tightly attaching the rods to the pedicle screws. Similar considerations can occur with screws other than pedicle screws in other applications in which the screws are utilized to support an implant construction, including, for example, applications in which the screws hold cages, plates, shafts and/or rods.

Various of the aspects discussed above for screws can also be applied to vertebral hooks. For instance, vertebral hooks can be formed to be porous; and /or to comprise, consist essentially of, or consist of a carbon/metal matrix, either alone, or coated with pyrocarbon. In exemplary aspects, such carbon/metal matrix can comprise, consist essentially of, or consist of carbon and tungsten. In other example embodiments, vertebral hooks can comprise any suitable pyrocarbon-coated material.

Exemplary vertebral hooks are shown in FIGS. 13-16 as hooks 500, 502, 504 and 506, respectively. The hook 506 is shown to have perforated regions. In other words, hook 506 is shown to have pores (which can be alternatively referred to as slots), similar to those described previously relative to the screws. The perforated regions can be utilized for enhancing union of the hook to a skeletal region, in a manner similar to that discussed above relative to the screws.

It is to be understood that the invention can include other porous structures besides those specifically shown in the drawings.

The specific aspects of the invention shown in the drawings and described above are but some exemplary aspects of the present invention. It is to be understood that the invention can also include other skeletal support structures comprising pyrocarbon-coated structures; or alternatively comprising, consisting essentially of, or consisting of a carbon/metal matrix, either alone, or coated with pyrocarbon. For instance, pyrocarbon-coated carbon/metal matrix materials can be utilized in rods, hooks, screws, vertebral spacers, vertebral replacement structures, and any other implant in which a material having high strength to weight ratio is desired. In some embodiments, a spacer provided between vertebrae to promote fusion (i.e., a cage) or preserve mobility (i.e., an artificial disk) can be formed of a pyrocarbon-coated carbon/metal matrix; a replacement vertebral body can be formed of a pyrocarbon-coated carbon/metal matrix; and/or a bridge utilized to bridge multiple vertebrae can have one or more components formed of a pyrocarbon-coated carbon/metal matrix.

Vertebral implants formed of pyrocarbon-coated carbon/metal matrices can be configured for utilization in any suitable procedure, such as, for example, posterior lumbar interbody fusion (PLIF), anterior lumbar interbody fusion (ALIF) and transforaminal lumbar interbody fusion (TLIF) procedures.

In other embodiments, joints and bones can be replaced with one or more components having pyrocarbon-coated carbon/metal matrices. For instance, ball joints, shoulders, elbows, hips, knees, facet joints, carpal bones, etc., can have one or more components having pyrocarbon-coated carbon/metal matrices. Such can include, for example, partial replacement of bones and/or joints; replacement of total bones and/or total joints; and/or bone-capping after amputation.

In some aspects, any therapeutic structure currently fabricated of a material other than a pyrocarbon-coated carbon/metal matrix (for instance, a structure currently fabricated from a titanium-containing material or a PEEK-containing material; where PEEK is poly(etheretherketone)) can instead be fabricated to comprise, consist essentially of, or consist of a pyrocarbon-coated carbon/metal matrix.

An example embodiment of an interbody spinal cage that can be formed of pyrocarbon-coated carbon/metal matrix is shown in FIG. 17 as a cage 600. The pyrocarbon-coated carbon/metal matrix can be of any of the types described previously in this disclosure. The cage has a plurality of orifices 602 extending therein (only some of which are labeled), has threads 604 (only some of which are labeled) for engaging vertebra, and has a tool engagement slot 606. The tool engagement slot enables a tool to rotate the cage and thereby screw the cage into place between a pair of vertebra. FIG. 18 shows another example interbody cage 610 engaging a pair of vertebra 612 and 614. The example cage 610 can also be formed of a pyrocarbon-coated carbon/metal matrix of any of the types described previously in this disclosure. Although cages 600 and 610 are described as being formed of pyrocarbon-coated carbon/metal matrices, it is to be understood that the cages could be formed of any suitable pyrocarbon-coated material. It is also to be understood that the cages could comprise carbon/metal matrices without pyrocarbon coating.

The cages 600 and 610 are example cages that can comprise pyrocarbon coating and/or carbon/metal matrices. It is to be understood that various aspects of the invention can include incorporation of pyrocarbon coating and/or carbon/metal matrices into any intervertebral cages, including cages which are not threaded. It is further to be understood that aspects of the present invention can be used for any intervertebral cages currently available, or which become available in the future. Also, it is to be understood that aspects of the invention can include incorporation of pyrocarbon coating and/or carbon/metal matrices into any therapeutic structures configured to replace spinal regions and/or to be attached with spinal regions, including, for example, spinal cages, spinal spacers, plates, replacement discs, and replacement vertebra.

As discussed throughout this document, pyrocarbon-coated carbon/metal matrices can be of benefit for utilization in numerous therapeutic devices. However, pyrocarbon-coated carbon/tungsten matrices can be of particular benefit for utilization in therapeutic structures configured to be associated with spinal regions, which can include structures that support or replace spinal regions. Specifically, the spine is subjected to enormous forces, and structures that support or replace spinal regions are also subjected to such enormous forces. Further, the forces are dynamic, changing both in direction and magnitude during common events, such as lifting, walking, falling, etc. Thus, it is desired to have an exceptionally strong biocompatible material to utilize for structures that support spinal regions (such as plates, rods and interbody cages), or replace spinal regions (such as replacement discs and replacement vertebrae). Pyrocarbon-coated carbon/tungsten matrices are extremely strong, with compression testing of materials containing about 10 weight % tungsten showing the materials to be stronger than conventional materials. This can enable less material of pyrocarbon-coated carbon/tungsten matrices (as opposed to conventional materials) to be utilized to form structures which still have desired strength for utilization for replacement of spinal regions or supporting spinal regions. This can enable thinner or otherwise smaller structures to be formed from pyrocarbon-coated carbon/tungsten matrices, which may improve patient comfort relative to conventional materials.

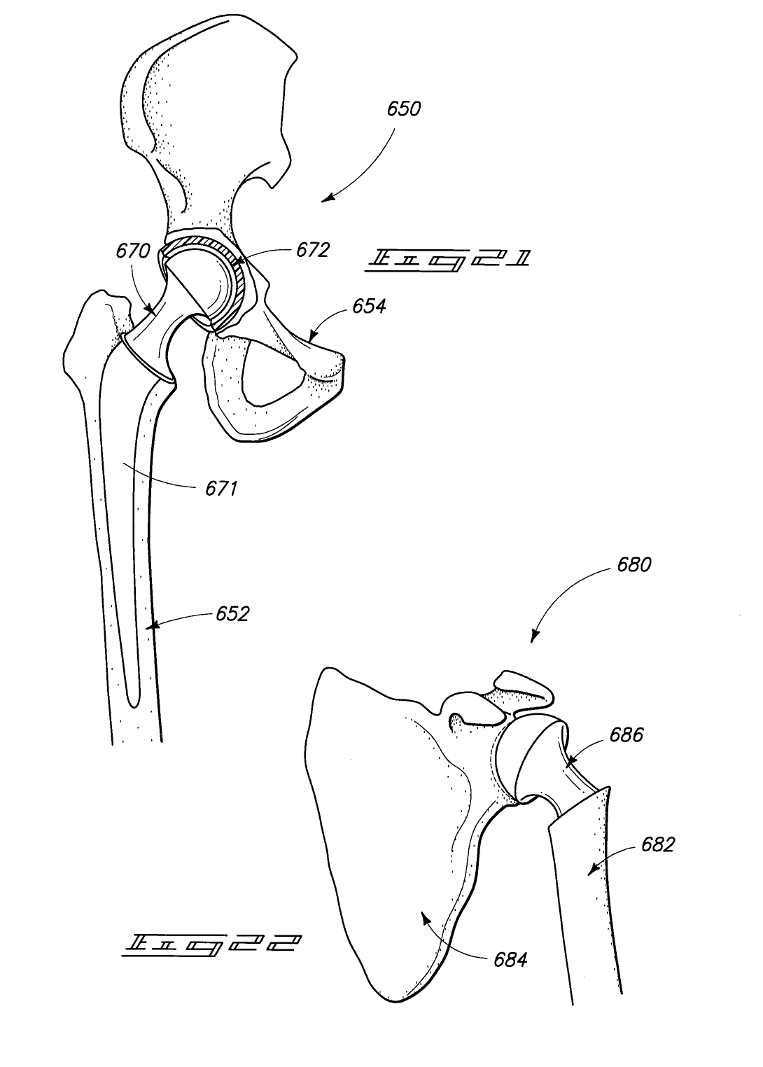

An example embodiment of a spinal spacer that can be formed of pyrocarbon-coated carbon/metal matrix is shown in FIG. 19 as a spacer 620. The pyrocarbon-coated carbon/metal matrix can be of any of the types described previously in this disclosure. FIG. 20 shows the spacer 620 retained against a vertebra 630. Although spacer 620 is described as being formed of a pyrocarbon-coated carbon/metal matrix, it is to be understood that the spacer could be formed of any suitable pyrocarbon-coated material. It is also to be understood that the spacer could comprise a carbon/metal matrix without pyrocarbon coating.

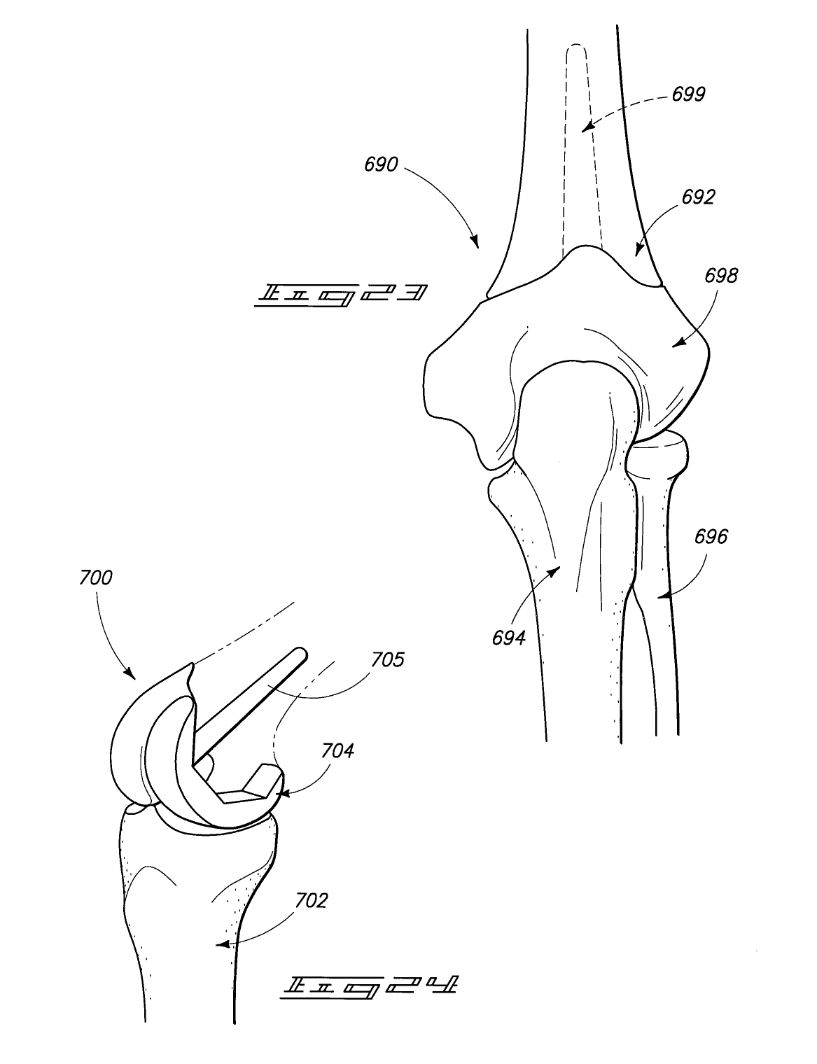

Example joints having portions replaced with pyrocarbon-coated carbon/metal matrix material are shown in FIGS. 21-24.

FIG. 21 shows a hip joint 650 comprising an example embodiment of the invention. The hip joint is comprised by a ball and socket, with the ball being joined to the femur 652 and the socket being joined to the innominate bones of the pelvis 654. However, the original ball of the femur is replaced by a prosthetic 670, and the original socket of the pelvis is replaced by a prosthetic 672. A stem 671 of the prosthetic 670 is diagrammatically extending downwardly into the femur. Either or both of the prosthetics 670 and 672 can be formed of a pyrocarbon-coated carbon/metal matrix of any of the types described previously in this disclosure. The prosthetics can be joined to adjacent supporting bones by conventional methods, such as, for example, screws and cements. For instance, the stem 671 of prosthetic 670 can be fastened to the femur with one or more screws; and/or with cement. Although only portions of the femur and pelvis are replaced by prosthetics in the shown aspect, it is to be understood that the entirety of one or both of the femur and pelvis could be replaced by prosthetic in other aspects (not shown). Although the prosthetics are described as being formed of pyrocarbon-coated carbon/metal matrices, it is to be understood that the prosthetics could be formed of any suitable pyrocarbon-coated material. It is also to be understood that the prosthetics could comprise carbon/metal matrices without pyrocarbon coating.

FIG. 22 shows a shoulder joint 680 comprising an example embodiment of the invention. The shoulder joint is comprised by a ball and socket, with the ball being joined to the humerus 682 and the socket being joined to the scapula 684. However, the original ball of the humerus is replaced by a prosthetic 686. The prosthetic can be formed of a pyrocarbon-coated carbon/metal matrix of any of the types described previously in this disclosure. The prosthetic can be joined to the humerus by conventional methods, such as, for example, screws and cements. All or part of the scapula can be replaced by a prosthetic comprising pyrocarbon-coated carbon/metal matrix in other embodiments (not shown). Although the prosthetics of the shoulder joint are described as being formed of pyrocarbon-coated carbon/metal matrices, it is to be understood that the prosthetics could be formed of any suitable pyrocarbon-coated material. It is also to be understood that the prosthetics could comprise carbon/metal matrices without pyrocarbon coating.

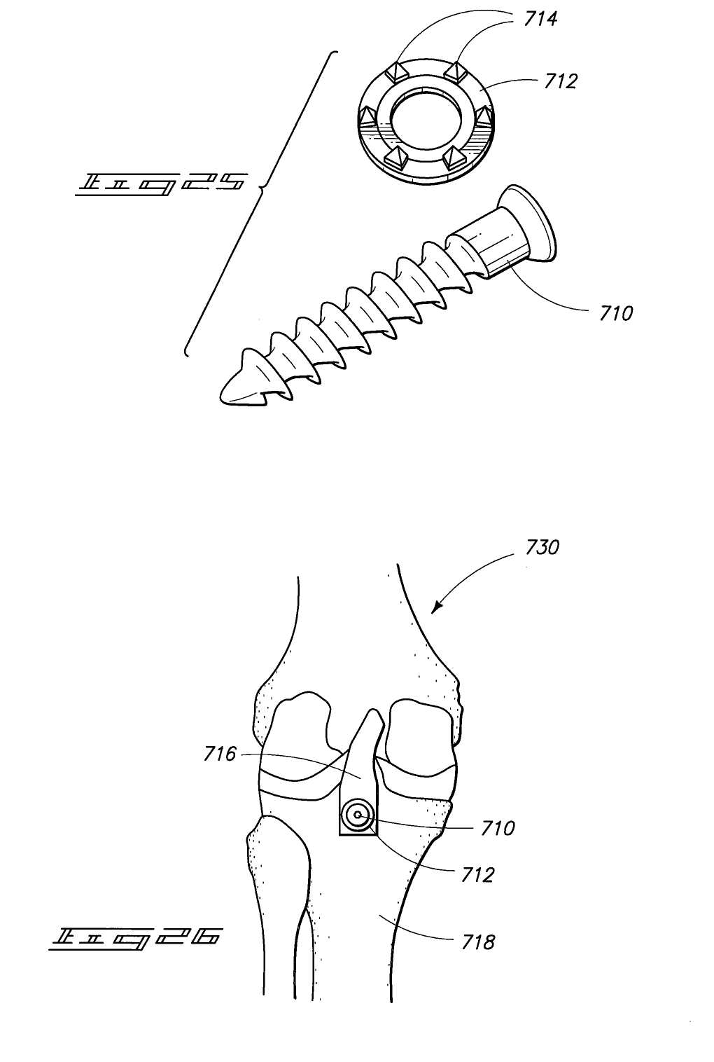

FIG. 23 shows an elbow joint 690 comprising an example embodiment of the invention. The elbow joint is comprised by humerus 692, ulna 694 and radius 696. In the shown embodiment, a lower portion of the humerus is replaced by a prosthetic 698. A stem 699 of the prosthetic is diagrammatically illustrated to extend upwardly into the humerus. The prosthetic can be formed of a pyrocarbon-coated carbon/metal matrix of any of the types described previously in this disclosure. The prosthetic can be joined to the humerus by conventional methods, such as, for example, screws and cements. All or part of one or both of the ulna and radius can also be replaced by prosthetics comprising pyrocarbon-coated carbon/metal matrix in other embodiments (not shown). Although the prosthetics of the elbow joint are described as being formed of pyrocarbon-coated carbon/metal matrices, it is to be understood that the prosthetics could be formed of any suitable pyrocarbon-coated material. It is also to be understood that the prosthetics could comprise carbon/metal matrices without pyrocarbon coating.

FIG. 24 shows a knee joint 700 comprising an example embodiment of the invention. The natural knee joint is comprised by the tibia and femur, but in the aspect of FIG. 24, the tibia is replaced with a prosthetic 702, and a portion of the femur is replaced with a prosthetic 704. The remaining femur is diagrammatically illustrated in dashed-line view. A stem 705 of the prosthetic 704 is diagrammatically extending upwardly into the femur. The prosthetic can be formed of a pyrocarbon-coated carbon/metal matrix of any of the types described previously in this disclosure. The prosthetics can be joined to supporting bones by conventional methods, such as, for example, screws and cements. Although the prosthetics of the knee joint are described as being formed of pyrocarbon-coated carbon/metal matrices, it is to be understood that the prosthetics could be formed of any suitable pyrocarbon-coated material. It is also to be understood that the prosthetics could comprise carbon/metal matrices without pyrocarbon coating.

The knee of FIG. 24 and elbow of FIG. 23 can be considered to be hinge joints of major limbs (with the major limbs being the leg and arm, respectively).

In some aspects, a patient can be an amputee missing the tibia and part of the femur, and the prosthetic 704 can be a bone cap provided after amputation of the lower part of the femur.

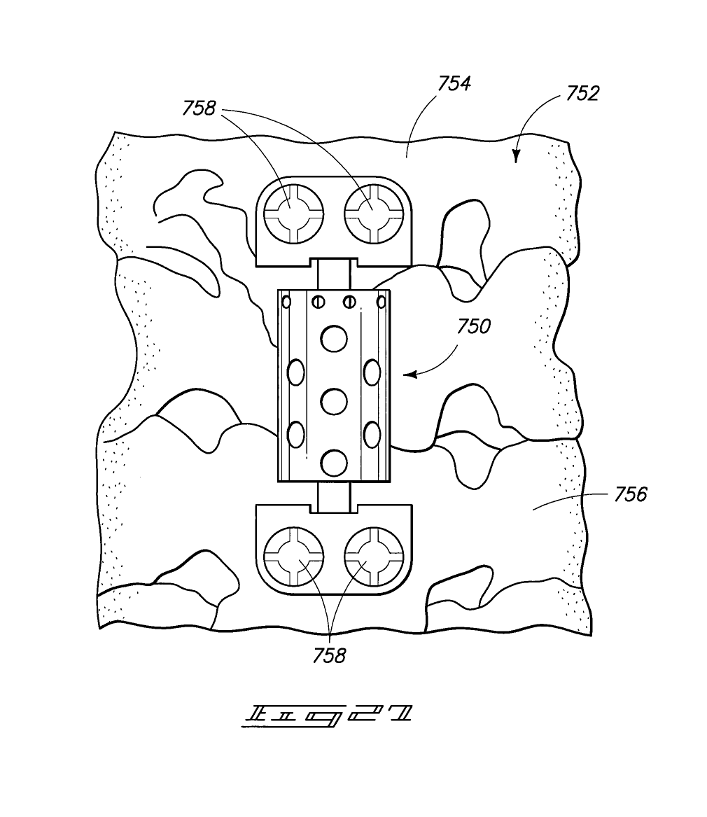

FIG. 25 shows a screw 710 and washer 712 that can be together utilized for attaching a tendon or ligament to a bone. The washer comprises a plurality of projections 714 (only some of which are labeled). Such projections are configured to engage the ligament or tendon. Either or both of the screw and washer can comprise pyrocarbon-coated material, and/or can comprise a carbon/metal matrix. In some embodiments, one or both of the screw and washer will comprise pyrocarbon-coated carbon/tungsten.

FIG. 26 shows a knee joint 730, and shows screw 710 and washer 712 together retaining a tendon 716 to a bone 718. The embodiment of FIGS. 25 and 26 is but one embodiment of a system configured to engage tendon or ligament to bone. Persons of ordinary skill in the art will recognize that there are numerous different systems available for engaging tendon or ligament to bone, some of which use screws and washers, some of which use screws alone, and some of which use other anchor structures alternatively, or in addition to, screws. In various embodiments, any of the components of a system configured to engage tendon or ligament to bone can be made of pyrocarbon-coated material; and/or can be made of a material comprising a carbon/metal matrix.

FIG. 27 shows a vertebral replacement embodiment 750 engaging a spine 752. The vertebral replacement structure 750 is retained to a pair of vertebra 754 and 756 with fasteners 758. The fasteners can be screws of the type described previously in this disclosure. Accordingly, in some aspects the fasteners can comprise pores suitable for bone to grow into the fasteners and help retain the fasteners.

The vertebral replacement structure 750 can comprise pyrocarbon-coated material, and/or can comprise a carbon/metal matrix. In some embodiments, the vertebral replacement structure will comprise pyrocarbon-coated carbon/tungsten. Persons of ordinary skill in the art will recognize that there are numerous vertebral replacement structures available. In various embodiments, any vertebral replacement structure available now, or which becomes available in the future, can be made of pyrocarbon-coated material; and/or can be made of a material comprising a carbon/metal matrix.

In compliance with the statute, the invention has been described in language more or less specific as to structural and methodical features. It is to be understood, however, that the invention is not limited to the specific features shown and described, since the means herein disclosed comprise preferred forms of putting the invention into effect. The invention is, therefore, claimed in any of its forms or modifications within the proper scope of the appended claims appropriately interpreted in accordance with the doctrine of equivalents.

1. A therapeutic structure configured to be associated with a spine, and comprising a pyrocarbon-coated material.

2. The structure of claim 1 configured to replace a portion of the spine.

3. The structure of claim 1 being an interbody cage.

4. The cage of claim 3 comprising a pyrocarbon-coated matrix; wherein the matrix comprises carbon and tungsten.

5. The cage of claim 4 wherein the matrix consists of carbon and tungsten.

6. The plate of claim 5 wherein the matrix comprises from about 1 weight % tungsten to about 30 weight % tungsten.

7. The structure of claim 1 being a spinal spacer.

8. The spacer of claim 7 comprising a pyrocarbon-coated matrix; wherein the matrix comprises carbon and tungsten.

9. The structure of claim 1 being a spinal plate.

10. The plate of claim 9 comprising a pyrocarbon-coated matrix; wherein the matrix comprises carbon and tungsten.

11. The plate of claim 10 wherein the matrix consists of carbon and tungsten.

12. The plate of claim 11 wherein the matrix comprises from about 1 weight % tungsten to about 30 weight % tungsten.

13. The plate of claim 12 being a cervical plate.

14. The plate of claim 13 being less than 1 millimeter thick.

15. The structure of claim 1 being a vertebral hook.

16. The hook of claim 15 comprising one or more pores extending therein, with said one or pores being configured to receive bone structure grown from bone adjacent the hook to enhance union of the hook with the bone.

17. The hook of claim 16 having bone cement within at least one of said one or more pores.

18. The hook of claim 16 having bone-growth-stimulating material within at least one of said one or more pores.

19. The hook of claim 15 comprising a pyrocarbon-coated matrix; wherein the matrix contains carbon and tungsten.

20. The structure of claim 1 being a vertebral body replacement.

21. The vertebral body replacement of claim 20 comprising a pyrocarbon-coated matrix; wherein the matrix contains carbon and tungsten.

22. A therapeutic structure configured to be associated with a spine, and comprising a carbon/metal matrix.

23. The structure of claim 22 being an interbody cage.

24. The structure of claim 22 being a vertebral body.

25. The structure of claim 22 being a spinal spacer.

26. The structure of claim 22 being a spinal plate.

27-31. (canceled)

32. The structure of claim 22 being a vertebral hook.

33-42. (canceled)

43. A screw comprising a pyrocarbon-coated material.

44. A screw consisting of a carbon/metal matrix encapsulated with pyrocarbon.

45-46. (canceled)

47. A screw configured to directly engage a bone, the screw comprising:
a shaft that is at least partially threaded;
at least one pore extending into the shaft and configured to receive bone structure grown from the bone to enhance union of the screw with the bone; and
wherein the screw comprises a carbon/metal matrix.

48-62. (canceled)

63. An amputee bone cap consisting of a carbon/tungsten matrix encapsulated with pyrocarbon.

64. A therapeutic construction, comprising:
a segment of a spinal column comprising a pair of vertebrae and a disk between the vertebrae; and
a structure comprising a carbon/metal matrix and attached to each vertebra of the pair of vertebrae with fasteners.

65-79. (canceled)

80. A prosthetic configured for replacing at least a portion of a ball and socket joint; the prosthetic comprising a pyrocarbon-coated matrix; the matrix containing carbon and tungsten.

81-84. (canceled)

85. A prosthetic configured for replacing at least a portion of a hinge joint of a major limb; the prosthetic comprising a pyrocarbon-coated matrix; the matrix containing carbon and tungsten.

86-89. (canceled)

90. A system for attaching tendon or ligament to bone and comprising at least one pyrocarbon-coated structure.

91-93. (canceled)
